(19) United States
(12) Patent Application Publication
(10) Pub. No.: US 2024/0199794 A1
(43) Pub. Date: Jun. 20, 2024

(54) Monomers and Polymers with Pendant Aspirin and Compositions and Methods Thereof

(71) Applicant: University of Massachusetts, Boston, MA (US)

(72) Inventors: Jie Song, Shrewsbury, MA (US); Jing Zhang, Guangzhou (CN)

(21) Appl. No.: 18/553,600

(22) PCT Filed: Mar. 25, 2022
(86) PCT No.: PCT/US2022/021847

§ 371 Date: Oct. 2, 2023

Related U.S. Application Data
(60) Provisional application No. 63/173,477, filed on Apr. 11, 2021.

(51) Int. Cl. C08G 63/08; A61L 27/18; A61L 27/54; A61L 27/58; C07D 319/12

(52) U.S. Cl. CPC C08G 63/08; A61L 27/18; A61L 27/54; A61L 27/58; C07D 319/12; A61L 2300/41; A61L 2400/16; C08G 2230/00; C08G 2280/00

(57) ABSTRACT

The invention provides novel compounds and polymers with pendant aspirin, and degradable and biocompatible compositions, medical devices and implants, and methods of making and use thereof.

### PRIORITY CLAIMS AND RELATED APPLICATIONS

This application claims the benefit of priority to U.S. Provisional Application No. 63/173,477, filed Apr. 11, 2021, the entire content of which is incorporated herein by reference for all purposes.

### STATEMENT REGARDING FEDERALLY FUNDED RESEARCH OR DEVELOPMENT

This invention was made with government support under grant numbers AR055615 and GM088678 awarded by the National Institutes of Health. The Government has certain rights in the invention.

### TECHNICAL FIELD OF THE INVENTION

The invention generally relates to materials and polymers useful in medical and healthcare applications. More particularly, the invention relates to novel compounds and polymers with pendant aspirin groups, and degradable and biocompatible compositions, medical devices and implants thereof, as well as methods for their synthesis and use.

### BACKGROUND OF THE INVENTION

Degradable shape-memory polymers (SMPs), capable of being fixed into a temporary shape for minimally invasive surgical delivery and triggered to revert to a preprogrammed permanent shape by external stimuli, are attractive for applications as self-tightening sutures, smart tissue scaffolds and drug-releasing cardiovascular stents. Polylactides (PLAs), particularly amorphous PLAs, due to their well established and tunable in vivo safety profiles as resorbable implants and drug delivery vehicles, are attractive candidates for engineering thermal responsive degradable SMPs and tissue engineering scaffolds. Poor temporary shape fixing and slow/incomplete shape recovery around physiological temperature, along with the brittleness and poor handling characteristics of pure amorphous PLAs, however, have attracted efforts to improve their toughness and shape memory programming by covalent crosslinking with urethanes, blending with other polymers, or block copolymerization with polyethylene glycol or polyurethanes with/without inorganic additives. However, existing copolymerization approaches often involve significant compositional changes (>50% non-PLA components) of the polymer. (Delaey, et al. 2020 Advanced Functional Materials 30 (44), 1909047; Hardy, et al. 2016 Advanced Materials 28 (27), 5717; Lendlein, et al. 2002 Science 296 (5573), 1673; Xie, et al. 2015 ACS applied materials & interfaces 7 (12), 6772; Zheng, et al. 2006 Biomaterials 27 (24), 4288; Xu, et al. 2010 Proceedings of the National Academy of Sciences 107 (17), 7652; Hiljanen-Vainio, et al. 1996 Macromolecular Chemistry and Physics 197 (4), 1503; Mehrabi Mazidi, et al. 2018 Macromolecules 51 (11), 4298; Zhang, et al. 2017 Advanced Functional Materials 27 (5), 1604784; Balk, et al. 2016 Advanced drug delivery reviews 107, 136; Liu, et al. 2007 Journal of materials chemistry 17 (16), 1543.)

Meanwhile, applications of PLA-based degradable polymeric scaffolds for guided tissue regeneration have also revealed challenges of the excessive acidic degradation products (e.g., lactic acid) that could cause significant inflammatory cells infiltrations and even local tissue resorption (osteolysis). Current approaches for modulating local immune responses to biomaterial implants include inhibiting protein or macrophage adhesion by engineering hydrophilic anti-fouling surfaces, neutralizing acidic degradation products by blending PLA with basic bioceramics such as hydroxyapatite, and systemic administration or local delivery of non-steroid anti-inflammatory drugs (NSAIDs) such as aspirin and ibuprofen. The first two approaches involve significant changes in material properties of the implant, while the latter approach may exert an undesired inhibitory effect on the critical early stage of tissue repair as a result of suppressing the critical inflammation/healing cascade following acute injury. (Zhang, et al. 2019 Science translational medicine 11 (502), eaau7411; Xu, et al. 2020 Biomaterials Translational 1 (1), 33; Suganuma, et al. 1993 Journal of applied biomaterials 4 (1), 13; Gaharwar, et al. 2020 Nature Reviews Materials 5 (9), 686; Visalakshan, et al. 2019 ACS applied materials & interfaces 11 (31), 27615; Lebre, et al. 2017 Scientific reports 7 (1), 1; Yuan, et al. 2014 Biomaterials Science 2 (4), 502; Xie, et al. 2019 Cell & Bioscience 9 (1), 103; Pountos, et al. 2012 The Scientific World Journal 2012; Barry 2010 Veterinary and Comparative Orthopaedics and Traumatology 23 (06), 385; Lasola, et al. 2020 Frontiers in Immunology 11, 1726; Mountziaris, et al. 2011 Tissue Engineering Part B: Reviews 17 (6), 393.)

There is an urgent need for novel and improved strategies that can simultaneously enhance the mechanical such as toughness and handling properties as well as shape-memory performances of PLAs while selectively mitigating degradation-induced local inflammatory responses without perturbing the critical early-stage inflammation/tissue healing cascade.

### SUMMARY OF THE INVENTION

The invention is based on the unexpected discovery of novel compounds and polymers with pendant aspirin, and degradable and biocompatible compositions, medical devices and implants thereof, as well as methods of their making and use in various medical and healthcare applications. In particular, the invention provides a novel class of degradable SMPs with improved physical and biological properties. The dynamic hydrophobic interactions among the aspirin pendants efficiently modulate the thermal, mechanical, rheological, and energy dissipative properties of the copolymer in an aspirin incorporation content-dependent manner, resulting in gigapascal-storage moduli at body temperature, significantly modulated mechanical strength and enhanced toughness without significant alteration of their chemical compositions (e.g., with only ˜5 or 10% or less aspirin pendent incorporation) or polymer synthesis and processing.

The thermoplastic copolymers disclosed herein can be efficiently prepared by melt ring opening polymerization (ROP) from D,L-lactide and aspirin-functionalized monomer AspGA (e.g., 5-10%). The copolymers exhibited outstanding shape memory performance around a physiologically relevant temperature range, achieving nearly perfect temporary shape fixing at room temperature and facile shape recovery at above room temperature (e.g., 50° C.). The hydrolytic degradation products of these copolymers did not negatively impact the proliferation or osteogenesis of BMSCs in vitro. Additionally, the copolymers are far more resistant to fractures during materials handling compared to PLA control of comparable molecular weight, and when implanted subcutaneously in immune-competent rats, mitigated PLA degradation-induced inflammation by the concomitant hydrolysis of the aspirin pendants and the release of salicylic acid from the copolymer, without suppressing early acute inflammatory responses.

In one aspect, the invention generally relates to a compound having the structural formula (I):

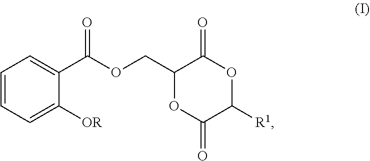

wherein R is H or Ac, and R¹ is H or an alkyl (e.g., C₁₋₃ alkyl) group.

In another aspect, the invention generally relates to a copolymer comprising a structural unit of formula (II):

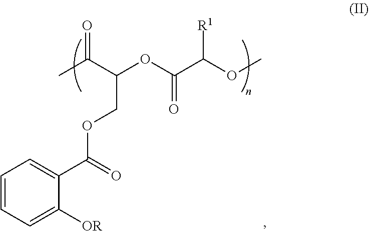

wherein R is H or Ac, R¹ is H or an alkyl (e.g., C₁₋₃ alkyl) group, and n is an integer from about 1 to about 200.

In yet another aspect, the invention generally relates to a copolymer comprising a structural unit of formula (III):

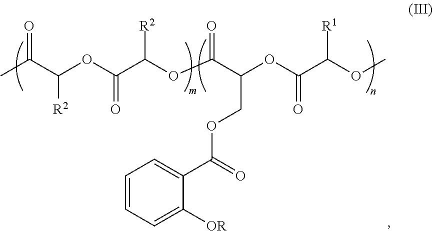

wherein R is H or Ac, R¹ is H or an alkyl (e.g., C₁₋₃ alkyl) group, R² is H or an alkyl (e.g., C₁₋₃ alkyl) group, and each of m and n is a positive integer (e.g., about 1 to about 200) with the ratio of m:n in the range of about 99:1 to about 80:20.

In yet another aspect, the invention generally relates to a composition comprising a copolymer of the invention.

In yet another aspect, the invention generally relates to a degradable material comprising a copolymer of the invention.

### BRIEF DESCRIPTION OF THE DRAWINGS

FIG. 1. (A) DSC thermograms (2nd heating cycle) of PLA, 5.9% poly(AspGA-co-LA) and 10.5% poly(AspGA-co-LA) from −40 ° ° C. to 180 ° C. at a 10 ° C. min⁻¹heating rate. (B) Thermal mechanical properties of 5.9% poly(AspGA-co-LA) vs. 10.5% poly(AspGA-co-LA) obtained on a Q800 DMA using a tensile fixture. (C) Storage/loss moduli and (D) complex viscosity as a function of rheological oscillatory temperature sweep of 10.5% poly(AspGA-co-LA), 5.9% poly(AspGA-co-LA) vs. the PLA control determined on an AR 2000EX rheometer at a scanning rate of 5 ° C. min⁻¹and a constant angular frequency of 1 rad s⁻¹.

FIG. 2. (A) Ultimate tensile stress/strain of 5.9% and 10.5% poly(AspGA-co-LA) determined on an MTS Bionix 370; (B)-(C) Consecutive (5) tensile loading/unloading of 5.9% and 10.5% poly(AspGA-co-LA) specimens on a Q800 DMA under force control (0 to 18 N to 0; 1 N min⁻¹); (D) Representative stress relaxation profiles of 5.9% and 10.5% poly(AspGA-co-LA) at 25° C. Inset: stress relaxation time (τ_{1/2}), N=3, **P<0.01 (Student's/text). (E)-(F) Stress relaxation of 5.9% and 10.5% poly(AspGA-co-LA) at different temperatures. Insets: respective activation energy determinations.

FIG. 3. One-way shape memory cycles of (A) 5.9% poly(AspGA-co-LA) and (B) 10.5% poly(AspGA-co-LA) along with the respective R_{f}'s at 25° C. and Rᵣ's at 65° C. determined from the second cycle. (C) Demonstration of shape recovery of 10.5% poly(AspGA-co-LA) from bent “temporary” shape upon heating at 50° C.

FIG. 4. In vivo degradation and drug release of polymers upon rat subcutaneous implantation. (A) Representative macroscopic images of each pellet (30 mg) before (left) and after (right) 16-week implantation. Scale bar=3 mm. (B) HPLC chromatographs (UV detector, 228 nm) of aspirin standard, salicylic acid standard and extract of explanted 10.5% poly(AspGA-co-LA) after 12-week implantation. (C) Representative H&E staining of 16-week explants at 200× and 500× magnifications (boxed areas of the top panels), with the implant remnant positioned on the right side of each optical micrograph. Scale bar=20 μm. BV=blood vessel; F=fibroblast; FBGC=foreign body giant cell; M=macrophage. (D) Quantification (n=3) of total numbers of FBGCs in five combined random fields of view at 400× magnification. *p<0.05 vs. PLA control (one-way ANOVA Tukey's multiple comparison).

FIG. 5. ¹H NMR spectra of monomer AspGA and polymer 10.5% poly(AspGA-co-LA) (top) and FTIR (bottom) spectra of 10.5% poly(AspGA-co-LA), 5.9% poly(AspGA-co-LA) and PLA control. Arrows indicate typical aromatic stretching/bending frequencies attributed to Aspirin pendants.

FIG. 6. Representative images of the water droplets on PLA control, 5.9% poly(AspGA-co-LA) and 10.5% poly(AspGA-co-LA) films and the measured water contact angles (N=6). ****P<0.001 vs. PLA control or indicated by the bracket (One-way ANOVA Tukey's multiple comparison).

FIG. 7. The brittle PLA control tends to break (A) while the more compliant poly(AspGA-co-LA) could be readily bent and twisted without breakage.

FIG. 8. Optical micrographs of a bisected 10.5 wt % poly(AspGA-co-LA) specimen before and after various time of thermal healing at 70° C.

FIG. 9. Mass residue (%) of polymers over time upon incubation in PBS (pH 7.4, 10 mg/mL) at 70° C. (N=3).

FIG. 10. Cytocompatibility of degradation products of PLA vs. poly(AspGA-co-LA). (A) Viability of rat BMSCs, as determined by CCK-8 assay (N=3), on days 1, 3 and 5 of culture in the expansion media with the supplementation of 12.5 ug/mL of PLA, 5.9% poly(AspGA-co-LA) and 10.5% poly(AspGA-co-LA) degradation products, respectively. ns: p>0.05, vs. PBS supplementation control at respective timepoints. (B) Representative Alizarin red staining of rat BMSCs after 2-week culture in osteogenic media with the supplementation of 12.5 μg/mL of PLA, 5.9% poly(AspGA-co-LA) and 10.5% poly(AspGA-co-LA) degradation products at every media change, respectively.

FIG. 11. A pilot study examining the temporal changes in inflammatory responses within the fibrous tissue capsules surrounding each implant at 2-, 8-, 12- and 16-week post-implantation. (A) Representative H&E staining of the explants over time. Scale bar=20 μm. (B) Quantification of average number of macrophages in five random FOVs at 400× magnification.

### DETAILED DESCRIPTION OF THE INVENTION

The invention provides a novel class of degradable, thermoplastic copolymers suitable for a wide variety of medical and healthcare applications.

The disclosed invention addresses several key limitations of amorphous PLA-based biomaterials, including brittleness, poor shape memory properties, and degradation-induced inflammation, by strategic copolymerization of a novel aspirin-functionalized glycolide monomer AspGA with D, L-lactides. For example, a small percentage (˜10% or less) of aspirin-functionalized glycolide, (S)-(3,6-dioxo-1,4-dioxan-2-yl)methyl 2-acetoxybenzoate (AspGA) was copolymerized with D, L-lactide to enhance the thermal processing, toughness, shape memory efficiency of the copolymer while mitigating local inflammatory responses upon its degradation.

Without being bound by the theory, the hydrophobic aggregation among the aspirin pendants strengthen the physical entanglement of the PLA chains in the glassy state while their dynamic dissociation provides an effective mechanism for energy dissipation under loading or heating, thereby enhancing its toughness, thermal healing and shape recovery. The hydrophobic interactions among the pendant aspirin units lead to the formation of loosely packed domains with relatively low binding energy to dynamically strengthen the glassy-state physical entanglement of the amorphous PLA chains, facilitating shape memory programming and improving the viscoelasticity and toughness of the resulting copolymer. Meanwhile, the hydrolytic degradation of the copolymer would result in simultaneous hydrolysis of the aspirin pendants to release salicylic acid (active metabolite of NSAID aspirin), thereby timely mitigating the inflammatory response elicited by the immunogenic acidic degradation products of PLA while avoiding premature drug release.

In one aspect, the invention generally relates to a compound having the structural formula (I):

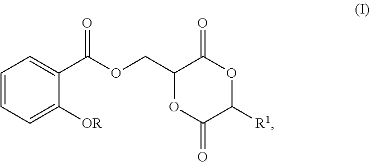

wherein R is H or Ac, and R¹ is H or an alkyl (e.g., C₁₋₃ alkyl) group.

In certain embodiments, the compound has the structural formula (Ia):

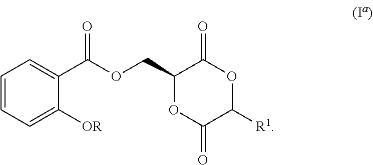

In certain embodiments, the compound has the structural formula (Ib):

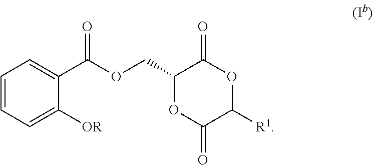

In certain embodiments of the compound, R is Ac.

In certain embodiments of the compound, R is H.

In certain embodiments of the compound, R¹ is H. In certain embodiments, R¹ is methyl.

In certain embodiments of the compound, the carbon to which R¹ is boned is in R-configuration. In certain embodiments, the carbon to which R¹ is boned R¹ is in S-configuration.

In another aspect, the invention generally relates to a copolymer comprising a structural unit of formula (II):

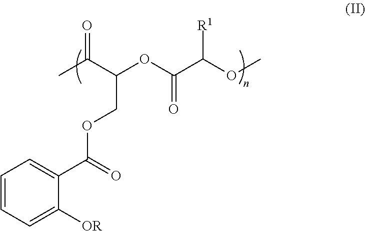

wherein R is H or Ac, R¹ is H or an alkyl (e.g., C₁₋₃ alkyl) group, and n is an integer from about 1 to about 200.

In certain embodiments of the copolymer, the copolymer has the structural formula (II^{a}):

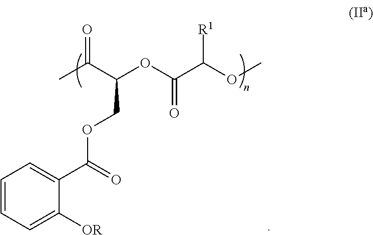

In certain embodiments of the copolymer, the copolymer has the structural formula

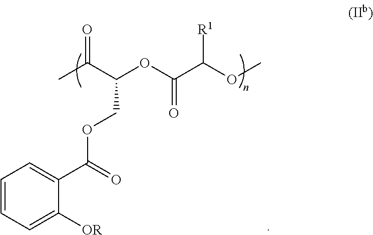

In certain embodiments of the copolymer, R is Ac.

In certain embodiments of the copolymer, R is H.

In certain embodiments of the copolymer, R¹ is H. In certain embodiments of the copolymer, R¹ is methyl.

In certain embodiments of the copolymer, the carbon to which R¹ is boned is in R-configuration. In certain embodiments of the copolymer, the carbon to which R¹ is boned R¹ is in S-configuration.

In certain embodiments, the copolymer further comprises a structural unit of ethylene glycol.

In certain embodiments, the copolymer further comprises one or more block of poly(ethylene glycol).

In yet another aspect, the invention generally relates to a copolymer comprising a structural unit of formula (III):

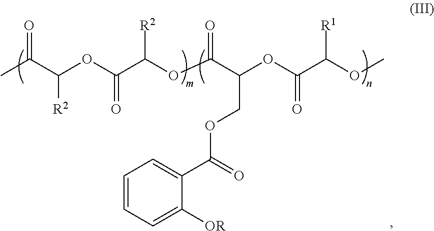

wherein R is H or Ac, R¹ is Hor an alkyl (e.g., C₁₋₃ alkyl) group, R² is H or an alkyl (e.g., C₁₋₃ alkyl) group, and each of m and n is a positive integer (e.g., about 1 to about 200) with the ratio of m:n in the range of about 99:1 to about 80:20.

In certain embodiments, the copolymer has the structural formula (III^{a}):

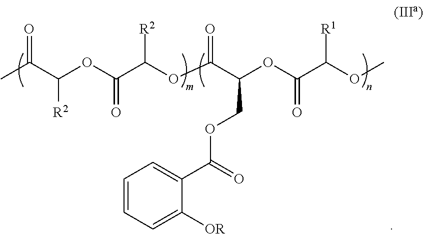

In certain embodiments, the copolymer has the structural formula (III^{b}):

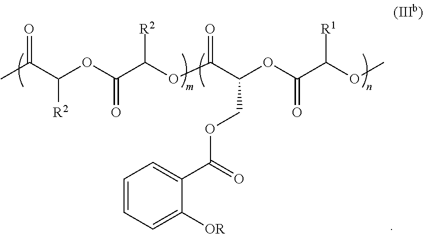

In certain embodiments of the copolymer, R is Ac.

In certain embodiments of the copolymer, R is H.

In certain embodiments of the copolymer, R¹ is H. In certain embodiments of the copolymer, R¹ is methyl.

In certain embodiments of the copolymer, R¹ is boned is in R-configuration. In certain embodiments of the copolymer, the carbon to which R¹ is boned R¹ is in S-configuration.

In certain embodiments of the copolymer, R² is methyl.

In certain embodiments, the copolymer further comprises a structural unit of ethylene glycol.

In certain embodiments, the copolymer further comprises one or more block of poly(ethylene glycol).

In certain embodiments of the copolymer, the ratio of m:n in the range of about 99:1 to about 80:20 (e.g., about 98:2 to about 80:20, about 95:5 to about 80:20, about 90:10 to about 80:20, about 99:1 to about 85:15, about 99:1 to about 90:10, about 99:1 to about 95:5, about 95:5 to about 90:10).

In certain embodiments, the copolymer has a Mn in the range of about 10,000 to about 200,000 (e.g., about 20,000 to about 200,000, about 50,000 to about 200,000, about 100,000 to about 200,000, about 10,000 to about 150,000, about 10,000 to about 100,000, about 10,000 to about 50,000).

In yet another aspect, the invention generally relates to a composition comprising a copolymer of the invention.

In yet another aspect, the invention generally relates to a degradable material comprising a copolymer of the invention.

As a proof-of concept example, a novel monomer AspGA was synthesized and copolymerized it with D,L-lactides in readily altered feed ratios by ROP. Using a rat subcutaneous implantation model, local immunological responses to the copolymers and PLA control prior to and during the scaffold degradation were investigated over 16 weeks. High-performance liquid chromatography and histological analysis of the explants were carried out to correlate the extent of scaffold degradation and drug release with the severity of local immune responses as a function of aspirin pendant content, supporting the mitigation of degradation-induced inflammation by the released salicylic acid.

Synergistically modulating mechanical properties and improving shape-memory performance while mitigating degradation-induced chronic inflammation of polylactide (PLA)-based implants for biomedical applications remains elusive. The hypothesis was tested that copolymerizing aspirin-functionalized glycolide with D, L-lactide could enhance the thermal processing, toughness, and shape memory efficiency of the copolymer while mitigating local inflammatory responses upon its degradation. The content of pendant aspirin was readily modulated by monomer feeds during ring opening polymerization, and the copolymers exhibited gigapascal-tensile moduli at body temperature and significantly improved fracture toughness and energy dissipation that positively correlated with aspirin pendant content. The copolymers also exhibited excellent thermal-healing and shape memory efficacy, achieving >97% temporary shape fixing ratio at room temperature and facile shape recovery at 50-65° C. These improvements were attributed to the dynamic hydrophobic aggregation among aspirin pendants that strengthened glassy-state physical entanglement of PLA while readily dissociate under mechanical loading/thermal activation. When subcutaneously implanted, the copolymers mitigated degradation-induced inflammation due to concomitant hydrolytic release of aspirin without suppressing early acute inflammatory responses. The incorporation of aspirin pendants in PLA represents a straightforward and innovative strategy to enhance the toughness, shape memory performance and in vivo safety of this important class of thermoplastics for biomedical applications.

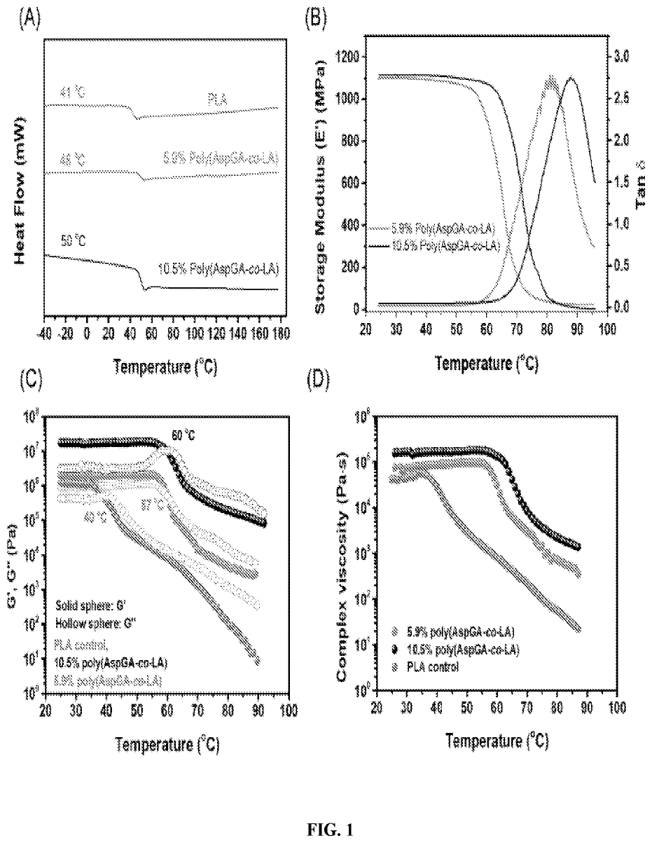

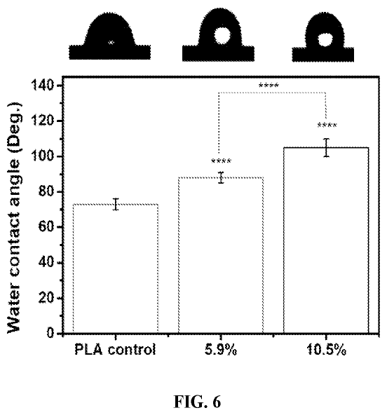

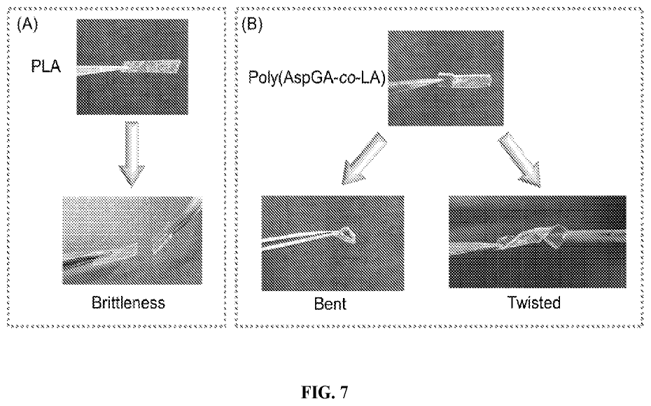

Using ROP, random copolymers poly(AspGA-co-LA) consisting of 0, 5.9, or 10.5% aspirin-bearing pendants with comparable molecular weights and polydispersity were synthesized. Water contact angle measurements confirmed that the incorporation of the aspirin pendants resulted in increased surface hydrophobicity. These thermoplastic polymers could be readily thermal-pressed into bulk specimens with various shapes and sizes, with the aspirin-functionalized copolymers exhibiting significantly improved ductility, toughness and handling characteristics (FIG. 7) desired for shape memory programming. DSC revealed an amorphous network structure free of crystalline domains for the copolymers, with an upward shift of T_{g} positively correlating with the AspGA content (FIG. 1A). Thermomechanical analyses revealed and validated the positive correlations of glassy-state modulus and T_{g} with the AspGA incorporation content, respectively (FIG. 6B). The gigapascal glassy-state storage moduli of poly(AspGA-co-LA), along with a T_{g} (48-50° C.) slightly above body temperature, make them uniquely suited for weight-bearing in vivo applications (e.g., as resorbable synthetic bone grafts). Oscillatory rheology experiments revealed a flow behavior characteristic for thermoplastic network where a high rubbery modulus plateau (FIG. 1C) and higher complex viscosity (FIG. 1D) were observed for both aspirin-functionalized copolymers, with an increasing AspGA incorporation content resulting in a more persistent solid-like behavior at higher temperatures. Overall, these thermal, mechanical and rheological properties collectively support the hypothesis that the hydrophobic interactions among the aspirin pendants augmented/stabilized the polymer chain entanglements, with these hydrophobic domains increasing T_{g}'s and enhancing the storage moduli and impeding the free-flowing of the polymer chains at the glassy state.

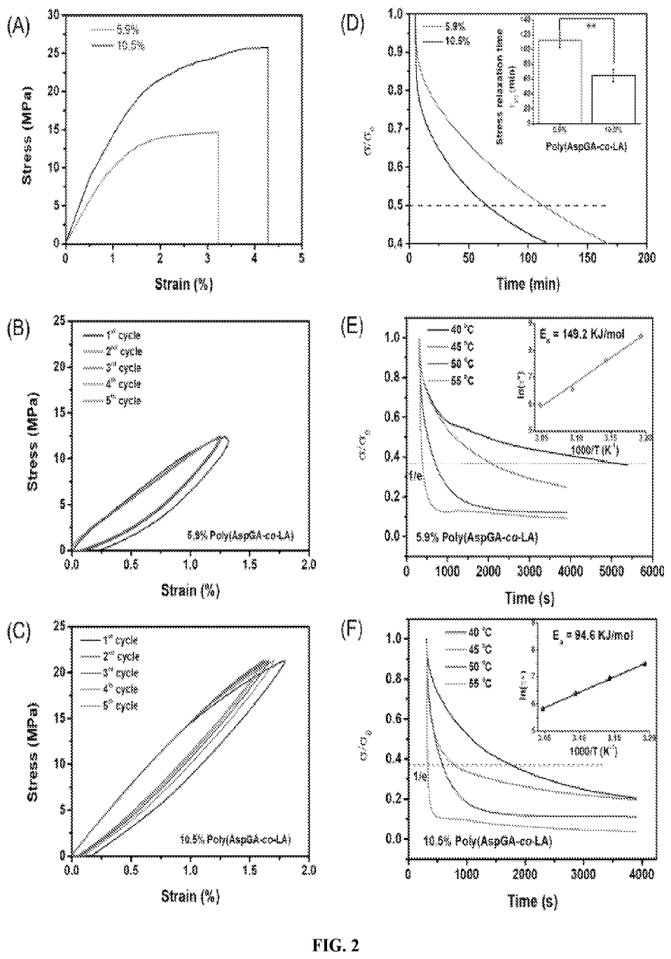

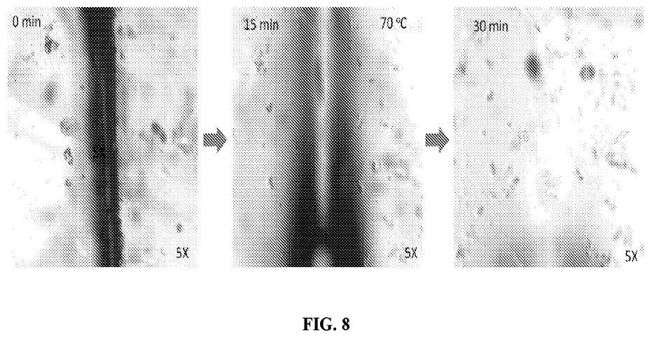

Furthermore, it was demonstrated that the incorporation of AspGA also resulted in significant improvements in the ultimate stress, ultimate strain, fracture toughness (FIG. 2A) and energy dissipative/stress relaxation properties (FIG. 2B-2D) of the copolymer in an aspirin pedant content-dependent manner (Table 3). These observations support that the hydrophobic interactions among the aspirin pendants are dynamic in nature, with their effective dissociation under stress providing an important toughening mechanism for the copolymers, which is attractive for their applications as shock-absorbing materials and weight-bearing medical implants. The more effective energy dissipation (FIG. 2B-2C) and faster stress relaxation (FIG. 2D) observed with the 10.5% poly(AspGA-co-LA) was further validated with a lower activation energy (Eₐ=94.6 KJ/mol) compared to the 5.9% poly(AspGA-co-LA) counterpart (Eₐ=149.2 KJ/mol; FIG. 2E-2F). It indicates that the dynamic disruption of the hydrophobic interaction among the aspirin pendants facilitates energy dissipation more effectively than PLA chain mobility and disentanglements. The low binding energy of aspirin pendant aggregations also translated into excellent thermal healing of the copolymer (FIG. 8), where the dynamic dissociation/association of the hydrophobic pendants along with enhanced PLA chain entanglement/disentanglement above T_{g} enabled the healing of the bisected specimens upon heating. This property can be exploited for the thermal repair/regeneration of poly(AspGA-co-LA) as structural materials and coatings.

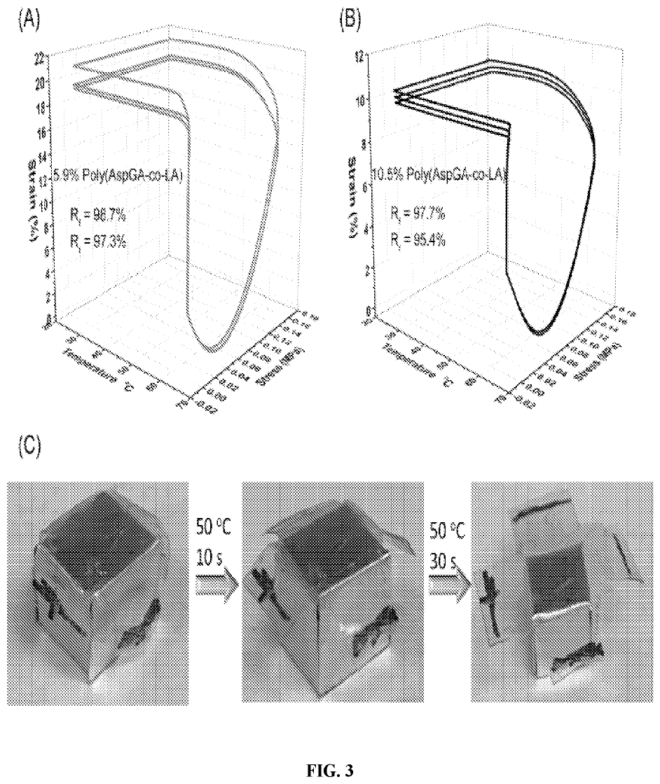

Finally, it was demonstrated that the excellent material handling characteristics (e.g. improved ductility and toughness), suitable thermal transition window (T_{g} near physiological temperature), and the hydrophobic interaction among the aspirin pendants dynamically strengthening PLA chain-chain interaction translated into outstanding shape memory performances. By simply incorporating 5-10% AspGA, nearly perfect temporary shape fixing at room temperature and facile shape recovery at 50-65° C. (FIG. 3) was achieved, a rare accomplishment with PLA-based thermoplastics without extensive changes of the polymer compositions or formulations.

In parallel, to establish the translational biomedical application potentials of these polymers, their hydrolytic degradation kinetics and the cytocompatibility of their degradation products were examined. Consistent with the increased surface hydrophobicity as revealed by water contact angles, the incorporation of aspirin pendants led to slower hydrolytic degradation of poly(AspGA-co-LA) compared to PLA control. Meanwhile, the slightly lower molecular weight of the 10.5% poly(AspGA-co-LA) resulted in a slightly faster hydrolytic degradation compared to 5.9% poly(AspGA-co-LA) despite the higher AspGA content of the former, underscoring both molecular weight and hydrophobicity as key factors influencing the polymer hydrolytic degradation. Continuous supplementation of the polymer degradation products (12.5 μg/mL) did not negatively impact the proliferation or osteogenesis of rat BMSCs, supporting good cytocompatibility of this class of degradable polymers and their potential applications as synthetic bone graft materials.

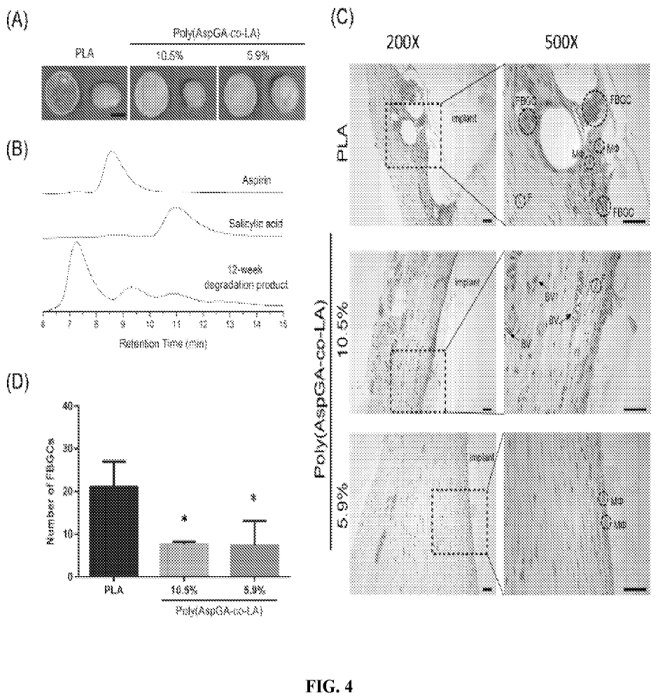

It was tested whether selective inhibition of the inflammatory response elicited by the in vivo degradation of PLA scaffolds, as opposed to the early acute inflammatory response required for initiating the healing cascade, can be accomplished by concomitant hydrolytic cleavage and release of covalently conjugated aspirin pendants from the synthetic copolymer. Gross inspection of the explanted pellet sizes of the explants (FIG. 4A) revealed that 5.9% poly(AspGA-co-LA), with almost identical molecular weight as the PLA control, exhibited slower in vivo degradation, consistent with its strengthened mechanical property and increased hydrophobicity, which have likely slowed water penetration and the hydrolytic degradation. The 10.5% poly(AspGA-co-LA), with slightly lower molecular weight than the PLA control, exhibited an overall in vivo degradation rate comparable to that of the PLA control. Histological examination of the explants showed that these implants elicited normal foreign body responses upon implantation and were minimally immunogenic prior to their degradation. However, at 16 weeks, when all explants had underwent substantial in vivo degradation (accompanied by the release of salicylic acid in the case of poly(AspGA-co-LA) (FIG. 4B), slightly lower numbers of macrophages and significantly less FBGCs (FIG. 4C-4D; Table 5) were detected within the fibrous tissue capsules surrounding poly(AspGA-co-LA). The degradation products elicited minimal hypersensitive/allergic responses (low counts of mast cells or eosinophils) and similar recruitment of neutrophils and lymphocytes (Table 5) across all formulations examined.

Macrophages are the primary immune cell type involved in both acute and chronic inflammatory responses, exerting either beneficial or deteriorating roles during the dynamic tissue regeneration process. (Luttikhuizen, et al. 2006 Tissue engineering 12 (7), 1955; Franz, et al. 2011 Biomaterials 32 (28), 6692.) In advanced stages of inflammation, macrophages undergo “frustrated phagocytosis” and fuse into FBGCs, which are known for higher degradative capacities than macrophages and could inhibit neovascular infiltration and tissue regeneration. (Zhao, et al. 1991 Journal of biomedical materials research 25 (2), 177; Xia, et al. 2006 Biomedical materials 1 (1), R^{1.}) Therefore, the detection of FBGCs within the fibrous tissue capsules surrounding biomaterial implants is often considered as a hallmark of poor biocompatibility or uncontrolled chronic inflammation. The mitigated immune responses detected within the fibrous tissue capsules surrounding poly(AspGA-co-LA) supported the hypothesis that concomitant hydrolytic cleavage of the pendant aspirin could mitigate scaffold degradation-induced inflammatory responses. Reduced number of FBGCs may also contribute to the slower immune cell-mediated degradation of the aspirin-conjugated polymers compared to PLA of similar molecular weight. For future studies, whether and how the temporally controlled local release of salicylic acid from the copolymers differentially impact macrophage polarization (e.g. M1 vs. M2 macrophages), which has been increasingly recognized for their complementary/opposing roles during various stages of inflammation and tissue repair, will be investigated. The efficacy of poly(AspGA-co-LA) in improving their in vivo performance as tissue engineering scaffolds needs to be validated using appropriate tissue defect models.

The following examples are meant to be illustrative of the practice of the invention, and not limiting in any way.

### EXAMPLES

### Monomer Synthesis, Copolymer Preparation and Characterizations

To accomplish a delayed release of anti-inflammatory drugs from PLA-based polymeric scaffolds and introduce hydrophobic pendants to facilitate their dynamic aggregation, a random copolymer bearing aromatic aspirin pendants was prepared through ROP of AspGA with D,L-lactide. To prepare the functional monomer AspGA (Scheme 1), lactone intermediate 4 was first prepared by acylation of an a-hydroxy acid (compound 2) and subsequent ring closure following the literature procedure. (Noga, et al. 2008 Biomacromolecules 9 (7), 2056; Leemhuis, et al. 2006 Macromolecules 39 (10), 3500.) The overall isolated yield of lactone 4 from O-benzyl-L-serine (compound 1) was almost 80% (20 g scale). The removal of the benzyl protection group was achieved by catalytic hydrogenation (Pd(OH)₂/C, 30% w/w in ethyl acetate) in 2 h, affording 5 in quantitative yield. The freed hydroxy group in 5 was used for subsequent conjugation with aspirin via esterification with catalytic amounts of 4-dimethylaminopyridine (DMAP) and stoichiometric acyl chloride. Functional monomer AspGA was obtained as white solid after recrystallization in a moderate isolated yield (33%). Protic solvents such as methanol were avoided throughout the synthesis as they could cause unintended ring-opening of the lactones.

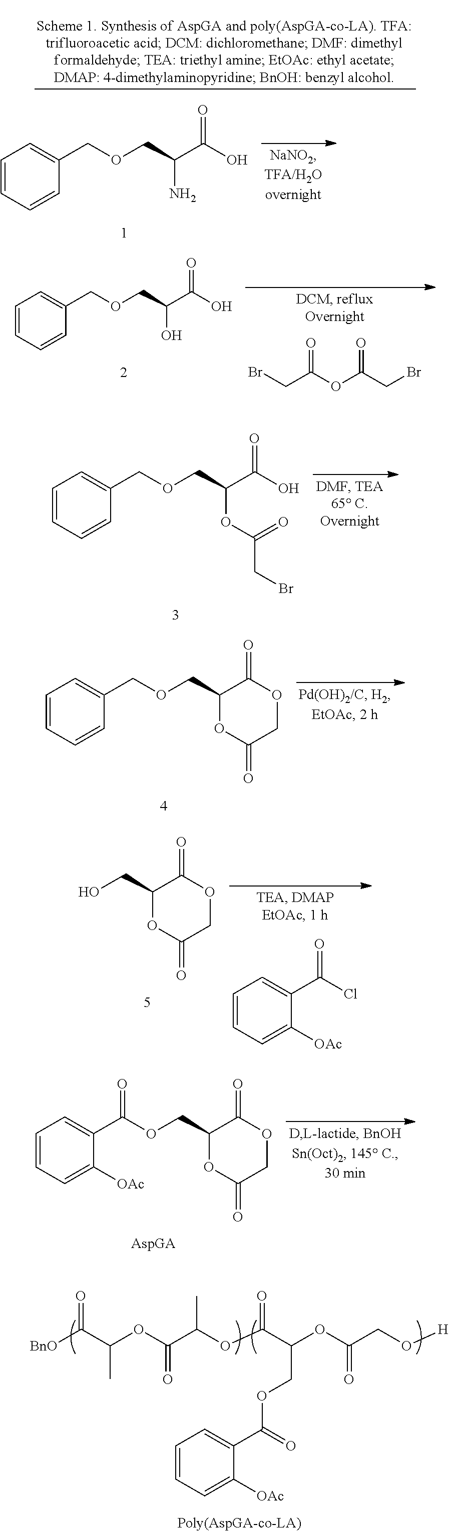

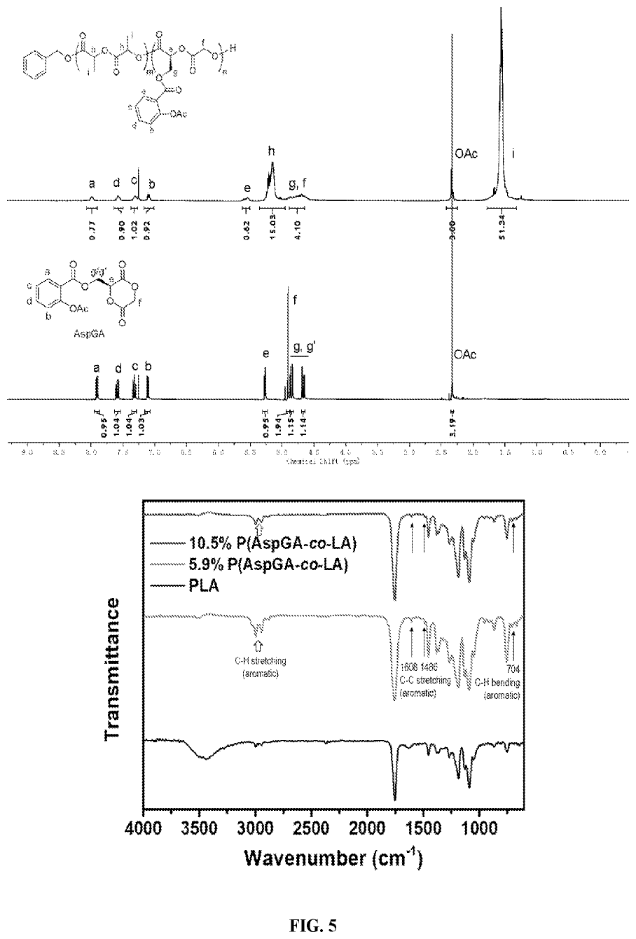

The random copolymers were prepared by melt ROP of AspGA with D,L-lactide using Sn(Oct)₂ as a catalyst and benzyl alcohol as an initiator at 145° C. with good monomer conversions (>85%). Meanwhile, PLA was also prepared by ROP as a control. ¹H NMR spectra of the purified polymers showed that the aromatic signals associated with aspirin were detected from the copolymers (FIG. 5), indicating that the aspirin moiety was well preserved during the melt ROP and subsequent workups. The aspirin pendant incorporation in polymers matched well with their respective monomer feeding ratios as determined by ¹H NMR integrations (Table 1). The successful incorporation of the aspirin pendants in the copolymers was further validated by FTIR (FIG. 5, bottom). Characteristic aromatic C—C stretching frequencies at 1608 cm⁻¹and 1486 cm⁻¹as well as aromatic C—H stretching and bending frequencies around the 2900-3000 cm⁻¹ region and at 704 cm⁻¹ regions were more prominently observed in both poly(AspGA-co-LA) specimens, ascribable to the aspirin pendants, 26than in the PLA (which only contained a trace aromatic initiator). GPC analysis (using polystyrene as standard) showed that relatively narrow polydispersity (Ð˜1.4) was obtained for copolymers incorporating 5.9% and 10.5% AspGA. A relatively close range of molecular weights (MW) was obtained for the PLA control (Mₙ=67800 g mol-1) and the poly(AspGA-co-LA) (Mₙ=67900 g mol⁻¹for 5.9% AspGA and Mₙ=46200 g mol⁻¹ for 10.5% AspGA). Water contact angle measurements of the polymer thin films formed on glass slides confirmed the expected increases in surface hydrophobicity upon aromatic aspirin pedant incorporation, with water contact angles increasing from 73° for PLA to 88° for 5.9% poly(AspGA-co-LA) and 105° for 10.5% poly(AspGA-co-LA) (FIG. 6). The obtained polymers were hot-pressed (Supporting Information) into pellets for mechanical tests and for in vivo subcutaneous implantation studies.

### Thermal Transitions and Thermomechanical Properties

Differential scanning calorimetry (DSC) of the copolymers revealed a narrow glass transition and no visible melt transition, supporting their largely amorphous nature. The copolymerization of AspGA resulted in an upward shift of glass transition temperature (T_{g}) compared to the PLA control in an AspGA content-dependent manner, with ΔT_{g}=7° C. for 5.9% poly(AspGA-co-LA) and ΔT_{g}=9° C. for 10.5% poly(AspGA-co-LA), respectively (FIG. 1A). The T_{g}'s of these poly(AspGA-co-LA), 48.5° C. and 50.3° C.(Table 2), are within a biologically safe range to trigger permanent shape recovery during shape memory programming. (Xu, et al. 2010 Proceedings of the National Academy of Sciences 107 (17), 7652.)

TABLE 2
Summary of thermal-mechanical properties of poly(AspGA-co-LA)

| AspGA (wt %) | T_{g}^{DSC} (° C.)^{a} | T_{g}^{DMA} (° C.)^{b} | Tanδ^{c} | E′_{37° C.}(MPa)^{d} | E′_{90° C.}(MPa)^{d} |
|---|---|---|---|---|---|
| 5.9 | 48.5 | 80.1 ± 0.8 | 2.73 ± 0.04 | 1097.9 ± 14.7 | 25.9 ± 14.7 |
| 10.5 | 50.3 | 85.2 ± 0.9 | 2.75 ± 0.05 | 1116.0 ± 27.5 | 6.75 ± 2.96 |

^{a}glass transition temperature determined from DSC curves.
^{b}glass transition temperature determined from Tanδ vs temperature curves.
^{c}peak value of Tanδ vs temperature curves.
^{d}storage moduli at 25° C. and 90° C. from DMA measurements, respectively.

Unlike the brittle PLA control that tended to break during thermomechanical molding or mounting on the DMA (FIG. 7A), the poly(AspGA-co-LA) copolymers exhibited good ductility. They could be bent and twisted without breaking (FIG. 7B), which is attractive for their shape memory programming. Thermomechanical analysis of the poly(AspGA-co-LA) specimens by dynamic mechanical analysis (DMA) confirmed the upward shift of T_{g}'s in an AspGA incorporation content-dependent manner (FIG. 1B). Both copolymers possessed high glassy-state storage moduli (>1-GPa) at body temperature (E′_{37° C.}, Table 2). This cortical bone-like mechanical strength may open the door for these copolymers as weight-bearing implants for in vivo orthopedic/dental applications. The slightly higher glassy-state tensile storage modulus of 10.5% poly(AspGA-co-LA) compared to 5.9% poly(AspGA-co-LA), despite a lower MW of the former, suggests that the hydrophobic interactions among the pendant aspirin units played an important role in strengthening the copolymer. The aggregation among the hydrophobic aspirin pendants have outweighed the relative reduction in PLA chain-chain entanglements below T_{g}'s in terms of the mechanical impact. In contrast, the storage modulus ate elastic state declined as the AspGA incorporation content increased while PLA content decreased, suggesting that the hydrophobically aggregated aspirin pendants were dissociated at the higher temperatures and the extent of physical entanglements of PLA was the predominant factor dictating the elastic storage modulus of the copolymers.

### Processing Rheological Properties

A key characteristic of dynamic polymer networks is their capability to flow under appropriate conditions. To better evaluate the influence of AspGA incorporation on the polymer viscoelastic characteristics and processability, oscillatory temperature sweep was performed (FIG. 1C). Above a characteristic crossover temperature (T_{crossover}) where storage modulus G′ equals to loss modulus G″, the material becomes fluid-like while below this temperature the material behaves like solids. Both poly(AspGA-co-LA) and the PLA control exhibited a rubbery plateau within the range of 10⁶-10⁸ Pa, with the rubbery moduli positively correlated with the AspGA incorporation content. T_{crossover} increased dramatically from 40° C. for PLA to 57° C. for 5.9% poly(AspGA-co-LA) and 60° C. for 10.5% poly(AspGA-co-LA), supporting that the presence of aspirin pendants impeded the network flow as the glassy-state physical entanglements of PLA chains were stabilized by the hydrophobic aggregation of the aspirin units. The complex viscosity, another critical indicator of polymer rheology, dynamic interaction and processability, was also determined by the temperature sweep experiments (FIG. 1D). All 3 polymers examined exhibited a step change in complex viscosity around their respective flowing temperatures, with their complex viscosity holding steady below the transition while steadily declining above the transition, which is characteristic of thermoplastic networks. The complex viscosity of 10.5% poly(AspGA-co-LA) below the flowing point was significantly higher than that of the copolymer with a lower AspGA incorporation content (5.9%) or the PLA control. These rheological data are in general agreement with the observations from the DSC and thermomechanical measurements, further supporting that the incorporation of aspirin pendants significantly augmented/stabilized the glassy-state PLA entanglements.

### Ultimate Tensile Stress Strain, Toughness and Energy Dissipation Properties

Uniaxial tensile test-to-failure experiments showed that the incorporation of higher content of AspGA (10.5%) led to significant enhancements in tensile modulus by >55% (to 1.74 GPa), ultimate strain by 30% (to 4.3%), ultimate stress by 70% (to ˜25 MPa), and tensile fracture toughness by 130% (to ˜82 MJ m⁻³) compared to 5.9% poly(AspGA-co-LA) (FIG. 2A; Table 3).

TABLE 3
Network parameters for poly(AspGA-co-LA).

| AspGA | Tensile | Strain | Tensile stress | Fracture | Dissipated | |
|---|---|---|---|---|---|---|
| content | modulus | at break | at break | toughness | energy | Eₐ |
| (wt %) | (MPa)^{a} | (%)^{a} | (MPa)^{a} | (MJ m⁻³)^{a} | (MJ m⁻³)^{b} | (KJ mol⁻¹)^{c} |
| 5.9 | 1120 ± 20 | 3.3 ± 0.1 | 14.6 ± 0.4 | 35.58 ± 3.1 | 4.64 ± 0.5 | 149.2 |
| 10.5 | 1744 ± 30 | 4.3 ± 0.1 | 24.8 ± 0.5 | 81.94 ± 6.3 | 7.19 ± 0.6 | 94.6 |

^{a}measured/calculated from the data obtained on MTS.
^{b}measured/calculated from the first cycle of consecutive tensile loading/unloading on DMA.
^{c}calculated by the Arrhenius equation τ(T) = τ₀ exp(Eₐ/RT) from tensile stress relaxation experiments at varied temperatures carried out on DMA.

Energy dissipations within poly(AspGA-co-LA) were investigated by five consecutive tensile loading and unloading cycles on the DMA (FIG. 2B-2C, Table 3). Hysteresis loops between the loading and unloading curves, resulting from the energy dissipation within the viscoelastic polymers, were observed for both compositions. Whereas the hysteresis loops for 5.9% poly(AspGA-co-LA) after the first cycle largely overlapped, those for the 10.5% poly(AspGA-co-LA) continued to shift. The dissipated energy, quantified as the area of the hysteresis loop, steadily dropped from 7.19±0.6 MJ m⁻³ (8.8% of the total input work) in the first cycle to 5.54±0.7 MJ m⁻³ in the second cycle and 4.04±0.5 MJ m⁻³ in the 5th cycle for 10.5% poly(AspGA-co-LA) (Table 4) under force-controlled loading/unloading (0 to 18 N to 0). In contrast, the 5.9% poly(AspGA-co-LA) dissipated 4.64±0.5 MJ m⁻³ energy (13% of the total input work) in the first cycle and 3.37±0.3 MJ m⁻³ in the second cycle and 3.03±0.2 MJ m⁻³ in the 5th cycle (FIG. 2B-2C, Table 4). Although more energy was dissipated in 10.5% poly(AspGA-co-LA) than in 5.9% poly(AspGA-co-LA), the lost energy represented a lower percentage of total input work, reflecting the much higher toughness (total energy absorbed) of the 10.5% poly(AspGA-co-LA). The steadier declines in the dissipated energy between the 1^{st} and 5^{th} cycles for the copolymer with the higher content of aspirin pendants also suggests that not all hydrophobic interactions were fully restored within the timeframe the cyclic experiments.

TABLE 4
Comparison of dissipated energy in consecutive
tensile loading-unloading cycles.

| AspGA | Dissipated energy (MJ m⁻³) | | | | |
|---|---|---|---|---|---|
| content | 1^{st} | 2^{nd} | 3^{rd} | 4^{th} | 5^{th} |
| (wt %) | cycle | cycle | cycle | cycle | cycle |
| 5.9 | 4.64 ± 0.5 | 3.37 ± 0.3 | 3.15 ± 0.3 | 3.07 ± 0.2 | 3.03 ± 0.2 |
| 10.5 | 7.19 ± 0.6 | 5.54 ± 0.7 | 4.80 ± 0.5 | 4.36 ± 0.4 | 4.04 ± 0.5 |

The viscoelastic 10.5% poly(AspGA-co-LA) was characterized with faster stress relaxation (T1/2) compared to its lower AspGA content counterpart (FIG. 2D and inset), suggesting that the dynamic association/dissociation among the aspirin pendants directly contributed to the energy dissipation within the polymer network. Finally, stress relaxation activation energy determined by the varying temperature experiments revealed much lower activation energy for 10.5% poly(AspGA-co-LA) (Eₐ=94.6 KJ/mol; FIG. 2F and inset) than its lower AspGA incorporation content counterpart (Eₐ=149.2 KJ/mol; FIG. 2E and inset) which had higher PLA content. These trends support that the dynamic hydrophobic interactions among the aspirin pendants were more effective in energy dissipation than that of the mobility and physical entanglement/disentanglement of PLA chains.

### Thermal-Healing Behavior

To test the hypothesis that the dynamic nature of the hydrophobic aspirin pendant interaction and PLA chain entanglements could translate into good thermal-healing, a 10.5% poly(AspGA-co-LA) specimen was bisected and examined for the ability of the re-opposed interface to heal above T_{g}. As shown in FIG. 8, the bisected specimen exhibited impressive healing across the interface within 30 min at 70° C., supporting that the enhanced PLA and aspirin pendants mobility at the higher temperature enabled the reestablishment of PLA physical entanglements and hydrophobic aspirin pendant interactions across the interface.

### Thermal Responsive Shape-Memory Behavior

The shape-memory performance of poly(AspGA-co-LA) was quantitatively assessed via stress-controlled one-way shape-memory cycles. At both AspGA incorporation contents examined, excellent temporary shape fixing ratio (R_{f}: 98.7% for 5.9% AspGA, 97.7% for 10.5% AspGA) at 25° C. and shape recovery ratio (Rᵣ: 97.3% for 5.9% AspGA, 95.4% for 10.5% AspGA) at 65° C. were achieved (FIG. 3A-3B). The outstanding temporary shape fixing ratios achieved by incorporating only 5-10% AspGA was attributed to the hydrophobic interaction/π-π stacking among the aspirin pendants that helped fix/stabilize the stretched PLA chains in the temporary shape. The dynamic nature of the hydrophobic interactions among aspirin pendants, on the other hand, ensured their timely dissociations upon heating to allow efficient recoiling of the stretched PLA chains to return to their elastic state. The more efficient shape recovery observed with 5.9% poly(AspGA-co-LA) was consistent with the fact that less hydrophobic interactions would have to be overcome during the shape recovery process. Finally, it was demonstrated the feasibility of achieving facile shape recovery under the safe triggering temperature of 50° C., where a bulk 10.5% poly(AspGA-co-LA) achieved good shape recovery within 30 seconds (FIG. 3C).

### In Vitro Hydrolytic Degradation and Cytocompatibility of the Degradation Products

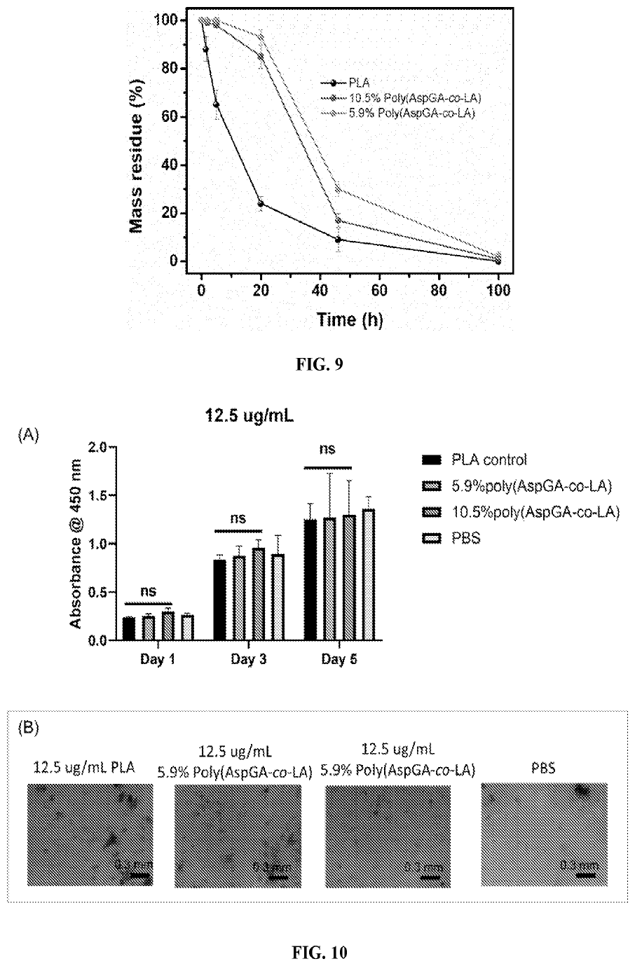

With the incorporation of hydrophobic aspirin pendants, slower hydrolytic degradations of the poly(AspGA-co-LA) was expected compared to PLA due to slower water penetration of the former. Indeed, accelerated in vitro hydrolytic degradation in PBS (pH 7.4, 10 mg/mL) at 70° C. confirmed slower weight losses for both 5.9% and 10.5% poly(AspGA-co-LA) compared to PLA, resulting in 50% weight losses in 40 h, 30 h and 15 h, respectively (FIG. 9). The relatively faster hydrolytic degradation observed with 10.5% poly(AspGA-co-LA) compared to its 5.9% counterpart was attributed to the slightly lower molecular weight of the former, supporting that the overall hydrolytic degradation rate can be impacted by both hydrophobicity and molecular weight of the polymers.

The in vitro cytocompatibility of the degradation products of poly(AspGA-co-LA) and PLA, obtained after full disintegration of the bulk polymer through accelerated hydrolytic degradation in PBS (pH 7.4) at 70° C., was examined with rat BMSCs. Supplementation of 12.5 μg/mL¹² of the respective degradation products every other day to the BMSCs culture in expansion media did not result in statistically significant impact to their proliferation over 5 days compared to those supplemented with PBS control (FIG. 10A). Although the cell viability trended higher in those supplemented with the degradation products of poly(AspGA-co-LA) vs. that of PLA, the differences were statistically insignificant. Similarly, we did not observe any negative impact on the osteogenesis of rat BMSCs upon supplementation (12.5 μg/mL, 3 times a week) of the respective degradation products over the course of 2-week osteogenic induction, as evidenced by robust alizarin red staining observed in all groups (FIG. 10B).

### In Vivo Polymer Degradation and Drug Release

The extent of in vivo degradation of the polymer pellets implanted subcutaneously in rats was first grossly inspected by changes in explanted pellet dimensions over time (FIG. 4A). The PLA control and the 10.5% poly(AspGA-co-LA) degraded faster, resulting in smaller explanted pellets at 16 weeks, whereas the 5.9% poly(AspGA-co-LA) exhibited a slower degradation. This observation was consistent with the in vitro hydrolytic degradation profiles of these polymers (FIG. 9). The in vivo drug release within the fibrous tissue capsules was verified by HPLC. As shown in FIG. 4B, HPLC traces of the methanol extract of the explanted 10.5% poly(AspGA-co-LA) pellet and its tissue capsule revealed a peak corresponding to that of salicylic acid, supporting that the conjugated drug was cleaved off the polymer scaffold and deacetylated as the bulk degradation of the polymer occurred. The peak detected around 9.4 min suggests that part of the deacetylation of aspirin, the active form of aspirin, may have occurred after its cleavage from the polymer side chain (Scheme 2).

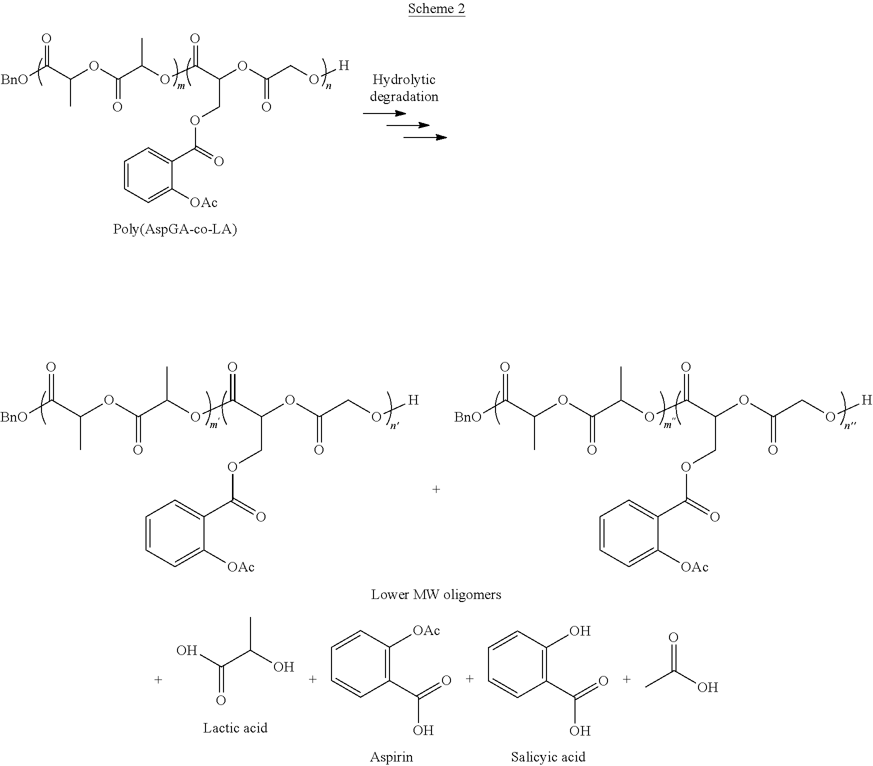

Likely hydrolytic degradation products of poly(AspGA-co-LA). n′, n″<n; m′, m″<m.

### Degradation-Induced Inflammatory Responses

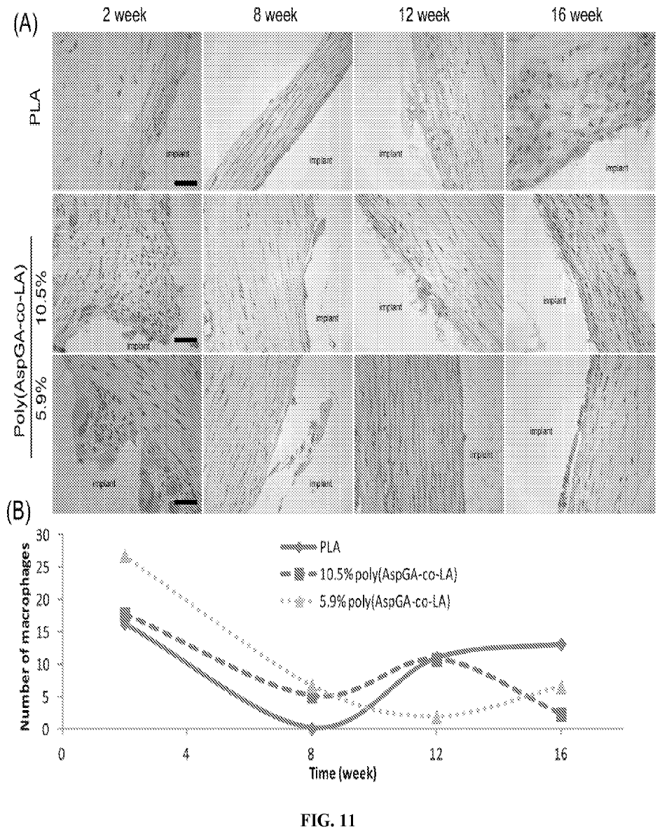

The pilot subcutaneous implantation data first revealed a similar foreign body response to the implantation of all polymer pellets as characterized with the fibrous tissue encapsulation (FIG. 11A). The initial acute inflammatory response observed at 2 weeks post-implantation subsidized over time resulting in a reduced number of immune cells (macrophages selectively quantified in the pilot study) detected within the fibrous tissue capsule and less active cellular activities by 8 weeks post-implantation (FIG. 11A-11B). These observations are consistent with literature reports and prior observations with the temporal changes of foreign body responses to other biomaterial implants prior to their degradation. (Filion, et al. 2011 Biomaterials 32 (4), 985; Williams 2008 Biomaterials 29 (20), 2941.)

The in vivo degradation of the pellets, however, triggered a secondary inflammatory response as characterized with increased cellular activities within the fibrous tissue capsules at 12 weeks post-implantation for the faster-degrading PLA and 10.5% poly(AspGA-co-LA), and at 16 weeks post-implantation for the slower-degrading 5.9% poly(AspGA-co-LA), respectively (FIG. 11A-11B). Notably, at 16 weeks, the secondary acute immune responses triggered by the degradation products subsidized surrounding 10.5% poly(AspGA-co-LA) while those surrounding the PLA control continued to exacerbate, suggesting a positive role of the in vivo released drug in mitigating the inflammatory response to degradation. To more thoroughly quantify the severity of the degradation-induced inflammatory responses as a function of the polymer composition, three 16-week explants of each composition were subjected to blind-evaluation by a pathologist. As shown in FIG. 4A, all the explanted pellets were encapsulated with a network of fibrous tissue. Quantification of immune cells within the fibrous tissue capsules (FIG. 4C) revealed less, although not statistically significant, macrophages within the fibrous tissue capsules surrounding poly(AspGA-co-LA) at 16 weeks compared to PLA (Table 5). Little difference was detected in the number of mast cells, eosinophils, neutrophils or lymphocytes across the three explanted polymer compositions. Significantly reduced number of foreign body giant cells (FBGCs, FIG. 4D), however, was detected within the fibrous tissue capsules surrounding the aspirin-incorporated polymers compared to the PLA control at 16 weeks, suggesting that the salicylic acid released at this stage effectively mitigated the inflammatory responses to scaffold degradation. No statistically significant difference in the degree of immune responses, however, was detected between the 5.9% vs. 10.5% poly(AspGA-co-LA).

TABLE 5
Quantification of inflammatory cells and
blood vessels in 16-week explants.^{a}

| | Macro- | Mast | Neutro- | Lympho- | | Blood |
|---|---|---|---|---|---|---|
| Implant | phage ^{b} | cell ^{c} | phil ^{c} | cyte ^{c} | Eosinophil ^{c} | vessel ^{d} |
| PLA | ++ | + | + | + | + | ++ |
| 10.5% | + | + | + | + | + | ++ |
| 5.9% | + | + | + | + | + | ++ |

^{a}Immune cells were counted in five randomly selected 400 × fields of view (FOV), and averaged.
^{b} For macrophage: <5/FOV (−), 5-10/FOV (+), 11-15/FOV (++), ≥16/FOV (+++)
^{c} For mast cell, neutrophil, lymphocyte, eosinophil: <2/FOV (+), 3-5/FOV (++), ≥5/FOV (+++).
^{d} For blood vessel: <5/FOV (+), 5-10/FOV (++), ≥11/FOV (+++).

### EXPERIMENTAL

### Materials

All chemicals were purchased from Sigma-Aldrich (St. Louis, MO) or Fisher Scientific (Pittsburgh, PA) and used as received unless otherwise specified. O-Benzyl-L-serine (99.8%) was purchased from Chem-Inpex International (Wood Dale, IL). D,L-Lactide was recrystallized twice with dry toluene prior to use.

### Monomer and Polymer Syntheses

### Bromoacetic Anhydride

Bromoacetic anhydride was synthesized per a previously reported literature. (Delaey, et al. 2020 Advanced Functional Materials 30 (44), 1909047.) In short, to a cold solution of bromoacetic acid (5.4 g, 38.6 mmol) in 10 mL of CH₂Cl₂ was added 1,3-diisopropylcarbodiimide (2.4 g, 19.3 mmol) diluted in 5 mL of CH₂Cl₂ slowly. Thereafter, the reaction mixture was stirred at room temperature for 1 h. The solution was filtered to remove the urea solid. Evaporation of the solvent from the filtrate provided bromoacetic anhydride (4.8 g, 90%) as yellow oil without further purification.

(S)-3-(benzyloxy)-2-hydroxypropanoic Acid (2)

Hydroxy acid derivative 2 was synthesized from O-benzyl-L-serine (1) per literature protocols. Briefly, compound 1 (20 g, 102 mmol) was added to an aqueous solution of trifluoroacetic acid (0.7 M, 293 mL) and stirred in an ice-bath until the white powder was totally dissolved. An aqueous solution of NaNO2 (14.5 g, 210 mmol, in 50 mL of H₂O) was then added slowly and the resulting yellowish solution was allowed to stir overnight before NaCl (20 g) was added. The mixture was extracted with ethyl ether three times, and the combined organic phase was washed with brine before being dried over anhydrous Na₂SO₄. After evaporating the solvent, spectroscopically pure yellowish liquid (20.10 g, 100%) was obtained. The ¹H NMR spectrum for 2 was in agreement with that reported in literature. (Hardy, et al. 2016 Advanced Materials 28 (27), 5717.)

(S)-3-(enzyloxy)-2-(2-bromoacetoxy)propanoic Acid (3)

Bromoacetic anhydride (2.9 g, 11.2 mmol) diluted in CH₂Cl₂ (5 mL) was slowly added to a cold solution of a-hydroxy acid (2.2 g, 11.2 mmol) in CH₂Cl₂ (20 mL). The mixture was then refluxed overnight. After cooling to room temperature, the mixture was diluted with CH₂Cl₂, washed with aqueous solutions of NaHCO₃ and brine. The organic layer was collected and purified by silica gel column chromatography with an eluent of hexane/ethyl acetate (2:1, v/v) to afford the product as a yellowish oil (3.2 g, 90%). ¹H NMR (500 MHz, Chloroform-d) 8 7.39-7.27 (m, 5H), 5.34 (dd, J=5.2, 2.8 Hz, 1H), 4.62 (q, J=12.2 Hz, 2H), 4.05-3.90 (m, 3H), 3.86 (dd, J=11.1, 2.8 Hz, 1H). 13C NMR (125 MHz, Chloroform-d) δ 171.76, 166.65, 137.06, 128.53, 128.50, 128.02, 127.82, 73.63, 73.08, 68.17, 25.19 ppm. The ¹H NMR spectrum for 3 was in agreement with that reported in literature. (Lendlein, et al. 2002 Science 296 (5573), 1673.)

(S)-3-((benzyloxy)methyl)-1,4-dioxane-2,5-dione (4)

Compound 3 (1.7 g, 5.4 mmol) dissolved in acetonitrile (20 mL) was added slowly to the acetonitrile solution containing triethylamine (TEA, 1.5 mL, 10.7 mmol) under the argon protection. The reaction mixture was stirred at 65° C. for 6 h. After cooling, the solvent was removed under reduced pressure, and the obtained crude product was purified by silica gel column chromatography with an eluent of hexane/ethyl acetate (2:1, v/v) to afford the product as a yellowish oil (1.1 g, 86%). ¹H NMR (500 MHz, Chloroform-d) δ 7.41-7.26 (m, 5H), 5.11 -5.01 (m, 2H), 4.81 (d, J=16.7 Hz, 1H), 4.58 (s, 2H), 4.08 (dd, J=10.5, 2.0 Hz, 1H), 3.96 (dd, J=10.5, 2.6 Hz, 1H) ppm. The ¹H NMR spectrum for 4 was in agreement with that reported in literature.

(S)-3-(hydroxymethyl)-1,4-dioxane-2,5-dione (5)

To a solution of compound 4 (900 mg, 3.81 mmol) in EtOAc (60 mL) was added Pd(OH)₂/C (30 wt %, 270 mg), and the mixture was subjected to cycles of purging to replace air with hydrogen. After stirring under hydrogen balloon for 2 h, the mixture was filtered through a short pad of silica gel to yield desired compound 5 (557 mg, 92.0%) as a colorless liquid. 1H NMR (400 MHz, d6-DMSO) δ 5.72 (t, J=4.8 Hz, 1H), 5.23 (t, J=3.2 Hz, 1H), 5.05 (m, 2H), 3.85 (m, 2H) ppm. ¹H NMR spectrum for 5 was in agreement with that reported in literature. (Zheng, et al. 2006 Biomaterials 27 (24), 4288.)

(S)-(3,6-dioxo-1,4-dioxan-2-yl)methyl 2-acetoxybenzoate (AspGA)

A solution of compound 5 (100 mg, 0.69 mmol) in EtOAc was stirred in an ice-bath for 10 min, before Et3N (0.19 mL, 1.37 mmol) and DMAP (17 mg, 0.14 mmol) were added. Five minutes later, a solution of acetylsalicyloyl chloride (272 mg, 1.37 mmol) in EtOAc was added dropwise. The white suspension was allowed to stir at room temperature for another 30 min before it was quenched with a cooled 5% NH₄Cl aqueous solution. The organic phase was washed with brine and dried over anhydrous Na₂SO₄. After evaporating the solvent, the resulting residue was purified by silica gel chromatography (n-hexane:EtOAc=1:1) to give a colorless liquid, which was further purified by recrystallization with Et₂O twice to afford compound AspGA as a white solid (33.3%). ¹H NMR (400 MHz, CDCl₃) δ 7.91 (dd, J=1.6, 8.0 Hz, 1H), 7.59 (m, 1H), 7.32 (m, 1H), 7.11 (dd, J=1.2, 8.0 Hz, 1H), 5.27 (dd, J=2.8, 4.8 Hz, 1H), 4.91 (d, J=2.8 Hz, 2H), 4.85 (dd, J=2.8, 12.8 Hz, 1H), 4.67 (dd, J=4.8, 12.8 Hz, 1H), 2.33 (s, 3H) ppm; 13C NMR (100 MHz, CDCl₃) δ 169.99, 163.84, 163.42, 163.22, 150.78, 134.83, 131.71, 126.51, 124.21, 122.54, 74.07, 65.61, 63.44, 21.19 ppm.

### Polymer Syntheses

The melt ring-opening polymerization (ROP) was conducted with different monomer feeding ratios to prepare the random copolymers and PLA control (Table 1).

TABLE 1
GPC and ¹H NMR characterizations of poly(AspGA-co-LA)

| | Aspirin percentage (%) | | | |
|---|---|---|---|---|
| | Theo- | Incor- | Mₙ^{GPC} | |
| Polymer | retical ^{a} | porated ^{b} | (g mol⁻¹) | Ð |
| 2% poly(AspGA-co-LA) | 2.87 | 2.03 | 74,900 | 1.74 |
| 4% poly(AspGA-co-LA) | 4.79 | 4.02 | 110,500 | 1.46 |
| 3% poly(AspGA-co-LA) | 3.00 | 3.03 | 119,600 | 1.44 |
| 5.9% poly(AspGA-co-LA) | 5.00 | 5.90 | 67,900 | 1.42 |
| 7.4% poly(AspGA-co-LA) | 8.00 | 7.35 | 63,000 | 1.43 |
| 10.5% poly(AspGA-co-LA) | 9.10 | 10.46 | 46,200 | 1.37 |

^{a} theoretical ratio based on molar percentage (mol %) of AspGA in the monomer feeds.
^{b} experimental molar percentage (mol %) of functional block length (n) in total polymer block length (n + m) based on ¹H NMR integrations.

In a typical procedure, the recrystallized AspGA (60 mg, 0.19 mmol) and D,L-lactide (908 mg, 6.30 mmol) were weighed into a dry Schlenk flask (10 mL), and the mixture was purged with argon three times. Benzyl alcohol (0.68 mg, 0.0063 mmol) and catalyst Sn(Oct)₂ (2.55 mg, 0.0063 mmol) in dry toluene stock solutions were then introduced via syringe injection. The reaction mixture was heated at 145° C. for 30 min. After cooling, the solidified mixture was dissolved in chloroform, precipitated in ice-cold methanol, and dried under vacuum to give the desired polymer (800 mg, 82.6%). ¹H NMR detection for residue D,L-lactide in crude product indicated a monomer conversion rate over 85%. GPC characterizations of the Mn and PDI of these polymers are summarized in Table 1. The final percentages of AspGA incorporation were calculated from ¹H NMR integrations based on the following equation:

$$\% ( AspGA ) = \frac{\int 2.324}{\int 2.324 + \frac{\int 1.56}{2}}$$

### Nuclear Magnetic Resonance (NMR) and Gel Permeation Chromatography (GPC)

NMR spectra were recorded on a Varian INOVA-400 spectrometer or a Bruker 500-MHz instrument. All small molecule compounds were visualized with 0.2 wt % orcinol in 2N H₂SO₄ for thin layer chromatography monitoring. Molecular weights and polydispersity of polymers were determined by gel permeation chromatography (GPC) with two 5-mm PLGel MiniMIX-D columns and a PL-ELS2100 evaporative light scattering detector and calibrated using polystyrene standards (Polymer Laboratories). THF was used as the eluent at a flow rate of 0.3 mL min⁻¹.

### Fourier-Transform Infrared (FTIR) Spectroscopy

FTIR spectra were recorded on a Nicolet IR 100 spectrometer (Thermo Electron Corporation) with 2-cm⁻¹ spectral resolution. The solid specimens were compressed into transparent discs with KBr.

### Water Contact Angle Measurements

Water contact angles on polymer films were measured with a CAM2000 goniometer (KSV Instruments) by the dynamic sessile drop method. Polymer thin films for water contact angle measurements were prepared by evaporation of 5% (w/w) chloroform solutions (0.1 mL) applied onto a clean glass slide at room temperature. After evaporating the first application of the chloroform solution for 10 min, another 0.1 mL of the polymer solution was applied and evaporated to achieve a thicker and more uniform coating. The water contact angles on the polymer thin films were measured at 25° C. A droplet (2 μL) of Milli-Q water was placed on the respective polymer thin films, and the contact angles of the droplet from left and right were recorded after 30 s. The reported contact angles were averaged from a total of six randomly selected regions (N=6) for each polymer.

### Material Fabrication and In Vitro Property Characterizations

### Polymer Pellet Fabrication

The bulk polymer specimens were prepared by hot pressing the as-synthesized polymer in a custom-made Teflon mold with a hydraulic press (CARVERR series NE, Wabash, IN). Briefly, Teflon film (0.008″ thick, Scientific Commodities Inc., Lake Havasu City, Arizona) was folded seven times, pressed tightly and carved or punched to create rectangular, dumbbell-shaped or cylindrical voids of specific dimensions. Then the as-prepared polymers, cut into small pieces, were placed into the voids, and pressed under a constant force of 2000 lbs at 120° C. for 5 min to fabricate specimens for various mechanical tests or in vivo implantations. ¹H NMR and GPC analysis indicated that there was no significant change in drug content or molecular weight of the polymers after hot pressing. For in vivo study, the obtained circular pellets (30.25±0.71 mg, 6.30 mm in diameter, 1.16 mm in thickness) were sterilized with 70% ethanol followed by UV irradiation (254 nm, 1 h each side) prior to implantation.

### Differential Scanning Calorimetry

DSC experiments were conducted on a Q200 MDSC (TA Instruments), which was calibrated with standard indium, at a heating rate of 10° C. min⁻¹ with a polymer specimen load of around 4.0 mg.

### Thermal Mechanical Properties

The dynamic mechanical properties of the poly(Asp-co-LA) as a function of AspGA incorporation content and temperature were determined on a Q800 Dynamic Mechanical Analyzer (TA Instruments) equipped with a gas cooling accessory and tensile film clamps. Specimens with dimensions of 10.0 mm×3.5 mm×0.3 mm were used for testing. The temperature was ramped from 25° C. to 100° C. at a heating rate of 5.0° C. min⁻¹. A 0.1% strain amplitude and 1.0 Hz frequency were applied. Three specimens (N=3) were tested for each polymer composition.

### Rheology

Complex viscosity and storage and loss moduli of the polymer formulations (˜20 mg) as a function of temperature sweep from 25 to 90° C. were determined by oscillatory rheology using an AR²⁰⁰⁰ EX rheometer equipped with 8 mm diameter parallel plates. The temperature scan rate was set to 5° C./min, and the frequency and strain was set to 1.0 rad/s and 1%, respectively.

### Tensile Test-to-Failure Tests

Tensile test-to-failure tests of poly(Asp-co-LA) as a function of AspGA incorporation content were carried out at room temperature on an MTS Bionix 370 servo-hydraulic mechanical tester equipped with a 250-N load cell. The test specimens, hot-pressed into dumbbell bones shape with a dimension of 28 mm in length, 3 mm in width and 1 mm in thickness, were mounted and strained to failure at 10 mm min⁻¹. The tensile modulus was calculated as the slope in the linear region (0-5%) of the stress-strain curve recorded. The fracture toughness was calculated by the area below the stress-strain curve.

### Energy Dissipation Properties and Stress Relaxation Measurements

Consecutive tensile loading and unloading (five cycles) of poly(Asp-co-LA) as a function of AspGA incorporation content were performed on a Q800 Dynamic Mechanical Analyzer (TA Instruments) equipped with a gas cooling accessory and tensile film clamps at 25° C., with the tensile force ramped from 0 to 18 N and back to 0 at a force ramping rate of 1 N min⁻¹, respectively. The dissipated energy was calculated as the area within the hysteresis loop of each tensile loading-unloading cycle.

The stress relaxation time (τ_{1/2}) of poly(Asp-co-LA) as a function of AspGA incorporation content (5.9% and 10.5%) and temperatures (40° C., 45° C., 50° C. and 55° C.) were determined on the Q800 Dynamic Mechanical Analyzer (TA Instruments) equipped with a gas cooling accessory and tensile film clamps under stress relaxation mode. All specimens were applied with a 1% constant strain and 1 Hz constant frequency while the load was recorded over time. The stress relaxation activation energy (Eₐ) is calculated by the Arrhenius equation τ(T)=τ₀ exp(Eₐ/RT), where τ(T) is the stress relaxation time at temperature T (K), τ₀ is a constant, and R is the ideal gas constant. τ(T) is deduced from the equation with reference to the respective relaxation curves per σ=σ₀ exp(−t/τ), where σ₀ is the initial stress, σ is the stress measured at time t, and the relaxation time τ is determined as the time when σ₀=1/e.

### Thermal Healing Property Demonstration

To demonstrate the thermal healing capability, a 10.5% poly(AspGA-co-LA) specimen (2 mm in length, 2 mm width, and 1 mm in thickness) was cut into two separate pieces using a razor blade. Subsequently, the separate pieces were brought back into contact and allowed to thermally heal at 70° C. for 30 min. The thermal healing at the broken and realigned interface was monitored by optical microscopy on a Leica M50 stereomicroscope equipped with a Leica DFC295 digital camera at 0, 15 and 30 min.

### Shape Memory Properties

One-way shape memory cycles were run on a Q800 Dynamic Mechanical Analyzer (TA Instruments) equipped with a gas cooling accessory and tensile film clamps. Specimens (10.0 mm × 3.5 mm × 0.3 mm) were first equilibrated at 85° C. for 10 min and then cooled down to 25° C.prior to testing. A preload force of 1 mN was applied to each specimen. Temperature was first raised to 65° C. (5° C. min⁻¹) and kept isothermal for 5 min. The specimen was deformed at 65° C. at a stress ramping rate of 0.02 MPa min⁻¹ from its “permanent” shape at the beginning of the N^{th} testing cycle, εₚ(N−1), to the elongated shape under a final tensile stress of 0.16 MPa. The temperature was then cooled to 25° C. with the stress kept constant at 0.16 MPa, and the sample length was recorded as ε₁ (N) (the strained sample length at the lower temperature at the Nth cycle). After being held at 25° C. for 5 min, the applied stress was released to the 1-mN preload force, and the sample length was recorded as εₚ(N) (the sample length at the lower temperature after unloading the tensile stress at the N^{th} cycle). Finally, the temperature was ramped from 25° C. to 65° C. at a heating rate of 5.0° C. min⁻¹ and kept isothermal for 5 min, and the final sample length was recorded as εₚ(N) [the recovered length at the N^{th} cycle or permanent length at the beginning of the (N+1)^{th} cycle]. The strain fixing ratio (R_{f}) and the strain recovery ratio (Rᵣ) in a given cycle N were determined by using the following equations:

$${R}_{f} ( N ) = \frac{ε u ( N ) - ε p ( N - 1 )}{ε l ( N ) - ε p ( N - 1 )}$$

$${R}_{r} ( N ) = \frac{ε u ( N ) - ε p ( N )}{ε u ( N ) - ε p ( N - 1 )}$$

### Accelerated In Vitro Hydrolytic Degradation of the Polymers

All specimens (N=3) were incubated in PBS (pH=7.4, 10 mg/mL) at 70° C. and retrieved at 1.5, 5, 20, 46 and 100 h. The retrieved specimens at each timepoint were washed with MilliQ water, freeze-dried, and weighed before being returned to an equal volume of fresh PBS for continued incubation at 70° C. until the next scheduled timepoint. The averaged percentage (%) of mass residue, defined as the dry mass at a given timepoint over the original dry weight of the specimen at time 0, was plotted over time for each polymer composition.

### Cyto-Compatibility Evaluations of the Hydrolytic Degradation Products of the Polymers

To determine the cytocompatibility of the degradation products of the respective polymers, each specimen was incubated in PBS (0.5 mg/mL) at 70° C. with frequent vortexing for 7 days, when no more bulk polymer was visible. The degradation solutions were sterile-filtered through 0.22-μm Teflon membrane before being supplemented to the expansion and osteogenic differentiation cultures of rat bone marrow derived stromal cells (BMSCs) at 12.5 μg/mL to determine their impact on the cell proliferation and osteogenesis. Passage 1 BMSCs, isolated from the tibia and femur of skeletally mature SASCO SD rats (male, 8-12 weeks) and enriched by plastic adherent culture, were used for these experiments.

For cell proliferation, BMSCs were plated at 2300 cells/cm2 and cultured in expansion media (αMEM without ascorbic acid, supplemented with 20% Hyclone characterized fetal bovine serum and 1% Pen-Strep). The filtered polymer degradation solutions were supplemented at a final concentration of 12.5 μg/mL every 2 days at the time of media change. Viability of cells at 1, 3, and 5 days were determined by CCK-8 assay (Dojindo, Japan), respectively. The absorbance was read at 450 nm with 620 nm background subtraction on a Multiskan FC Microplate Photometer (Thermo Scientific, Billerica, MA).

For F osteogenic differentiation, BMSCs was seeded at 25000/cm² and cultured in expansion media for 2 days (>70% confluency) before being switched to osteogenic media (expansion media supplemented with 10-nM dexamethasone, 20-mM β-glycerol phosphate, and 50-μM 1-ascorbic acid 2-phosphate) along with of the polymer degradation products (12.5 μg/ml). Media were changed 3 times a week for 2 weeks with the supplementation of the same concentrations of degradation products. After 2 weeks of osteogenic induction, cells were fixed with 10% formalin and stained with Alizarin red S for microscopic photo-documentation (100× magnification).

### In Vivo Studies

### Subcutaneous Implantation and Explant Retrieval

All animal surgeries were carried out according to the procedures approved by the University of Massachusetts Medical School Animal Care and Use Committee. Male SASCO-SD rats (289-300 g, Charles River Laboratories) were anesthetized with 5% isoflurane/oxygen and maintained under 2% isoflurane/oxygen throughout the surgery via a vaporizer. The rat abdomen was shaved, sprayed with 70% ethanol and wiped with Povidone-iodine. Following a small abdominal skin incision, a subcutaneous pocket was created by blunt dissection. The circular implant pellet (˜30 mg each) prepared from each of the three polymers shown in Table 1 was fit into each pocket and the incision was closed with wound clips. A total of seven specimens were implanted in a randomized fashion in each rat. Kefzol (20 mg/kg) and buprenorphine (0.08 mg/kg) were given immediately post-op. The rats were euthanized at predetermined time points and the fibrous tissue-encapsulated implants were retrieved. The explants were fixed in periodate-lysine-paraformaldehyde (PLP) fixative at 4° C. for 1 day and rinsed with PBS. In a pilot study, explants were retrieved at 2-, 8-, 12- and 16-week post-implantation for gross inspection of the degree of degradation and histological analysis, which revealed a rapid increase of inflammatory cells after 12 weeks as a result of implant degradation. For detailed histological analysis of immune cell infiltrations, a sample size of 3 was applied to the 16-week explants. In addition, one specimen from each group was retrieved at 8 weeks for SEM and at 12 weeks for HPLC detection of the drug release, respectively.

### High-Performance Liquid Chromatography (HPLC)

The in vivo degradation products of the 10.5% poly(AspGA-co-LA) within the explants were analyzed by reverse phase HPLC equipped with a Microsorb-MV 100-5 C18 250×4.6 mm column. Briefly, the 12-week explant was extracted with methanol for 2 days. The extract solution was filtered through a 0.2-μm polytetrafluoroethylene (PTFE) filter before being injected (20 uL) for HPLC. A mobile phase of methanol/1% aq. acetic acid (35/65) was applied at a flow rate of 0.7 mL/min and the signal was detected by UV at 228 nm. The obtained trace was compared with those of the aspirin and salicylic acid standard obtained under the same condition. The 12-week PLA explant was also analyzed to account for any background signals at the spectral regions of interest.

### Histological Analyses

Fixed explants were dehydrated in graded ethanol series and subjected to glycol methacrylate embedding and sectioning. Note that paraffin sectioning is not compatible for these polymers as they would dissolve during the deparaffinization. The 5-μm sections were stained with hematoxylin and eosin (H&E) for immune cell quantifications. The presence of macrophages, foreign body giant cells (FBGCs), mast cells, neutrophils, lymphocytes and eosinophils within the fibrous tissue capsules surrounding each implant was examined by a pathologist in a blinded fashion. Five randomly selected areas of the sections stained by H&E were used for quantification at 400× magnification.

### Statistics

All quantitative data are plotted or presented in tables as mean±standard derivation. Student's t-test was employed for pairwise comparisons in FIG. 2D inset and one-way ANOVA Tukey's multiple comparison was carried out for FIG. 4D and FIG. 6. Significance level was set as p<0.05.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I): 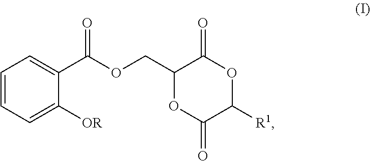
wherein R is H or Ac, and R¹ is H or an alkyl group.

2. The compound of claim 1, having the structural formula (I^{a}):

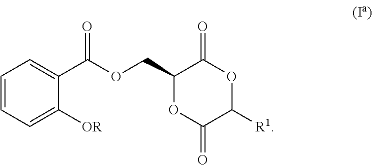

3. The compound of claim 1, having the structural formula (I^{b}):

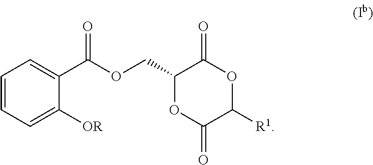

4. The compound of claim 1, wherein R is Ac.

5. The compound of claim 1, wherein R is H.

6. The compound of claim 1, wherein R¹ is H.

7. The compound of claim 1, wherein R¹ is methyl.

8. The compound of claim 7, wherein the carbon to which R¹ is boned is in R-configuration.

9. The compound of claim 7, wherein the carbon to which R¹ is boned R¹ is in S-configuration.

10. A copolymer comprising a structural unit of formula (II): 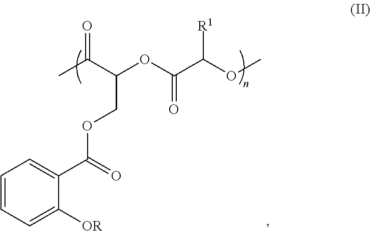
wherein R is H or Ac, R¹ is H or an alkyl group, and n is an integer from about 1 to about 200.

11. The copolymer of claim 10, having the structural formula (II^{a}):

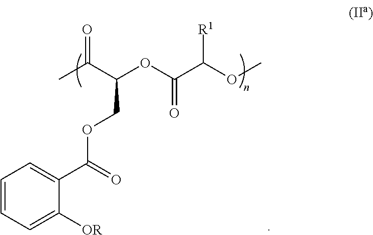

12. The copolymer of claim 10, having the structural formula (II^{b}):

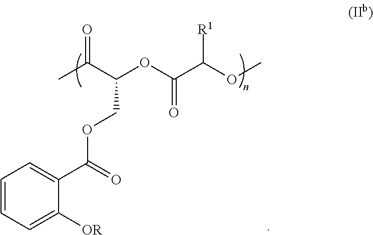

13-20. (canceled)

21. A copolymer comprising a structural unit of formula (III): 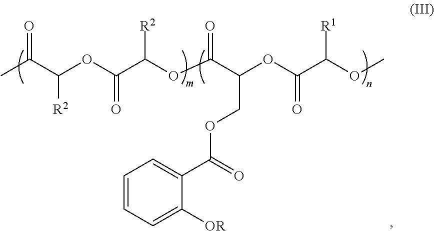
wherein
R is H or Ac,
R¹ is H or an alkyl group,
R² is H or an alkyl group, and
each of m and n is a positive integer with the ratio of m:n in the range of about 99:1 to about 80:20.

22. The copolymer of claim 21, having the structural formula (III^{a}):

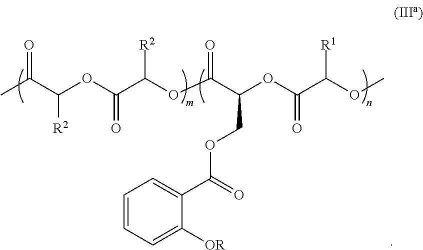

23. The copolymer of claim 21, having the structural formula (III^{b}):

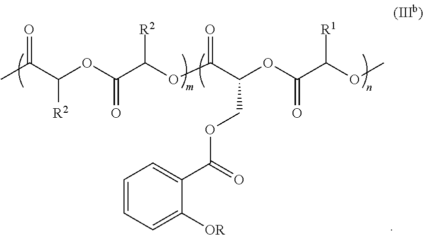

24-34. (canceled)

35. A composition comprising a copolymer of claim 10.

36. A degradable material comprising a copolymer of claim 10.

37. A composition comprising a copolymer of claim 21.

38. A degradable material comprising a copolymer of claim 21.
